(19) United States
(12) Patent Application Publication
(10) Pub. No.: US 2008/0102118 A1
(43) Pub. Date: May 1, 2008

(54) Glipizide controlled-release composition and method of preparation

(75) Inventors: Yong Gan, Huairou (CN); Xinteng Zhou, Huairou (CN)

Correspondence Address: BEYER WEAVER LLP, P.O. BOX 70250, OAKLAND, CA, 94612-0250, US

(73) Assignee: Ocean Star International, Inc., Snowville, UT (US)

(21) Appl. No.: 11/634,514

(22) Filed: Dec. 5, 2006

(30) Foreign Application Priority Data
Oct. 31, 2006 (CN) 200610114125.2

(51) Int. Cl. A61K 9/22

(52) U.S. Cl. 424/468

(57) ABSTRACT

A glipizide controlled release composition is provided comprising a drug-layer and a push-layer at a ratio of 1:0.5˜3 by weight. The drug-layer contains glipizide and 40˜99 percent by weight of the drug-layer of hydrophilic polyvinylpyrrolidone homopolymer and/or copolymer carrier. The push-layer comprises about 10 to 80 percent by weight of the push-layer of osmopolymers, about 10 to 80 percent by weight of the push-layer of water-insoluble polymers, and about 5 to 50 percent by weight of the push-layer of osmagents. The composition is used in osmotic pump tablets for controlled release of glipizide useful for administration once a day.

### 1. RELATED APPLICATION

Priority is claimed pursuant to 35 USC 119(a) from Chinese patent application Serial No. 200610114125.2, filed Oct. 31, 2006, incorporated by reference herein in its entirety.

This application is related to U.S. patent application Ser. No. ______ entitled “Nifedipine Controlled Release Compositions and Preparation Methods Therefor,” in the names of Yong Gan and Xinteng Zhou, filed concurrently herewith, and to U.S. patent application Ser. No. 11/580,215, filed Oct. 11, 2006, and U.S. patent application Ser. No. 11/599,150, filed Nov. 13, 2006, the disclosures of which are incorporated by reference herein in their entirety.

### 2. FIELD OF INVENTION

The present invention relates to the field of pharmaceutical preparation, to be precise it relates to an osmotic pump device containing the beneficial drug of glipizide with low solubility and its preparation method for controlled delivery of said beneficial drug thereof into the gastrointestinal tract at a controlled rate.

### 3. BACKGROUND OF THE INVENTION

Glipizide(N-[2-[4-[[[(cyclohexylamino)carbonyl]amino]sulfonyl]phenyl]ethyl]-5-methyl-pyrazinecarboxamide) is the second generation of sulfonyl ureas for lowering the blood glucose, mainly used for treatment of non insulin-dependent diabetes mellitus (NIDDM). According to the test data in vitro, the major mechanism of glipizide is to stimulate the beta cells of the pancreatic gland to secrete insulin, which shows an immediate action of lowering the blood glucose level. Furthermore, it has two important effects outside the pancreatic gland. It increases the sensitivity of target organs to insulin and decreases the glycogenesis in liver. The general oral formulation of glipizide is usually absorbed in the gastrointestinal tract quickly and completely, which leads to an immediate action of lowering blood glucose and a probable side effect of hypoglycemia. However, an oral controlled release formulation facilitates the administration, particularly administration just once a day, to control the blood glucose concentration at a constant level, which results in better compliance by patients and fewer side effects.

A commercial product, Glucotrol® XL (Pfizer), is a controlled release tablet formulation using a double-layer osmotic pump. Glipizide and hydrophilic inert ingredients are coated with semi permeable membrane. Glipizide is released from a laser-drilled orifice (as the tablet is swelling after absorbing water) at a controlled rate into the gastrointestinal tract, which is not affected by gastrointestinal peristalsis and pH. However, Glucotrol® XL contains polyethylene oxide(PEO) as the carrier of drug. We observe that use of PEO as the major drug carrier in an osmotic pump has some disadvantages. The PEO can cause a rather extensive time lag in distributing the drug because of its slow speed of water absorption and hydration, which makes the drug unable to take immediate effect. Also, the glass transition temperature (Tg) of PEO, typically in the range of 65° C. to 67° C., causes PEO to be not ideally heat stable. This can be problematic both in the preparation of the osmotic pump device and during storage. For example, it is difficult to remove solvent during the granulation process of tablet preparation. Since the granulation temperature is not usually above 40° C., the residue of organic solvent would be high or it would take an abnormally long period of time to properly dry. Furthermore, during tablet pressing, the temperature increases because of friction. When the temperature is above 50° C., conglutination may occur using PEO. Special equipment for cooling or for retarding the compacting speed during tablet pressing becomes necessary. Similarly, the stored temperature of a PEO carrier must be relatively low in order to retain its ideal drug release characteristics. Thus, the storage of the osmotic pump devices will typically require careful temperature control.

Accordingly, we provide a glipizide control release composition and osmotic pump tablet that obviates or alleviates these problems.

### 4. SUMMARY OF THE DISCLOSURE

The present invention provides a composition and an osmotic pump device for administration of the active pharmaceutical glipizide at a controlled rate into a biological environment. The device comprises a core comprising a first layer containing a pharmaceutically effective amount of glipizide and about 40 to 99 percent by weight of the first layer of carrier polymers comprising hydrophilic homopolymers and/or copolymers of polyvinylpyrrolidone, and a second layer comprising about 10 to 80 percent by weight of the second layer of water-insoluble polymers, about 80 to 10 percent by weight of the second layer of water-soluble osmopolymers, and about 5 to 50 percent by weight of the second layer of osmagents.

Preferably the ratio of weight of the first layer to the second layer is in the range of about 1:0.5 to 1:3.

Preferred polyvinylpyrrolidone homopolymers and copolymers are homopolymers of linear 1-vinyl-2-pyrrolidone groups and copolymers of 1-vinyl-2-pyrrolidone and vinyl acetate in the mass proportion of about 1:10 to 10:1.

The core of the device typically further comprises an adhesive, a lubricant, a glidant, and/or a colorant.

Particularly useful water-insoluble polymers for the second layer are selected from the group consisting of sodium starch glycolate, low-substituted hydroxypropyl cellulose, crosslinked carboxylmethyl cellulose sodium and mixtures of two or more thereof. Useful osmopolymers are selected from the group consisting of acrylic acid polymers, acrylic acid copolymers, hydroxypropylmethyl cellulose, polyvinypyrrolidone homopolymers, polyvinylpyrrolidone copolymers and mixtures of two or more thereof. Preferred acrylic acid polymers and/or copolymers comprise homopolymers of acrylic acid, crosslinked with an allyl ether pentaerythritol, allyl ether of sucrose, or allyl ether of propylene.

Useful osmagents for the second layer are selected from the group consisting of water soluble inorganic salts, organic acids, saccharides and mixtures of two or more thereof. Typical salts are selected from the group consisting of sodium chloride, potassium chloride, magnesium chloride, potassium sulphate, sodium sulphate and magnesium sulphate. Typical saccharides are selected from the group consisting of mannitol, sorbitol, xylitol, glucose and sucrose. Typical acids are selected from the group consisting of ascorbic acid and tarraric acid.

The device further comprises a wall surrounding the core comprising a semi-permeable material permeable to the passage of an exterior fluid and substantially impermeable to the passing of glipizide. The semi-permeable material typically comprises cellulose polymers. The wall accommodates a passageway communicating with the first layer and the exterior of the device for delivery of glipizide from the device. The passageway is usefully of the diameter of about 0.2 to 1.2 mm.

A method of preparation of the core of the device is provided comprising the steps of:

preparing the composition for the first layer by

a) separately passing glipizide, the carrier polymers and optional ingredients desired for the first layer through a 60-mesh sieve;

b) mixing the glipizide with the polymer carriers and the optional ingredients for the first layer to form a first ingredient mixture;

c) spraying the first ingredient mixture with an alcohol solution to form a first wet mixture;

d) granulating the first wet mixture; and preparing the composition for the second layer by
i) separately passing the water-insoluble polymers, water-soluble osmopolymers, osmagents and optional ingredients through a 60-mesh sieve;
ii) mixing the osmopolymers, water-insoluble polymers, osmagents and optional ingredients for the second layer to form a second ingredient mixture;
iii) spraying the second ingredient mixture with alcohol solution to form a second wet mixture;
iv) granulating the second wet mixture;
   1) pressing at least a portion of the granulated first wet mixture from step (d) to form the first layer;
   2) applying at least a portion of the granulated second wet mixture from step (iv) onto the first layer from step (1) to form a core precursor;
   3) pressing the core precursor to form the core comprising the first and second layers.

The method may further comprise the step 4) of coating the core with a membrane of semi-permeable polymers. The semi-permeable polymers typically comprise cellulose polymers. The optional ingredients of the second layer typically comprise an adhesive, lubricant, glidant, and/or colorant.

The method may further comprise the step of applying an anti-damp film over the membrane.

### 5. BRIEF DESCRIPTION OF THE DRAWINGS

FIG. 1 shows the curves of mean blood concentration to time of a single dose of a glipizide controlled release tablet made according to the present invention compared to a commercial product as described in Example 10.

FIG. 2 shows the curves of the logarithm of mean blood concentration to time of a single dose of a glipizide controlled release tablet made according to the present invention compared to a commercial product as described in Example 10.

### 6. DESCRIPTION OF THE PREFERRED EMBODIMENTS

The present invention provides a composition and osmotic pump which contain the active pharmaceutical glipizide for controlled release into the gastrointestinal tract.

The present invention also provides a method to preparing glipizide-containing controlled release tablets.

The present invention provides a controlled release composition of glipizide comprising a first layer (drug layer) containing a pharmaceutically effective amount of glipizide and about 40 to 99 percent by weight of the first layer of carrier polymers comprising hydrophilic homopolymers and/or copolymers of polyvinylpyrrolidone, and a second layer (push layer) comprising about 10 to 80 percent by weight of the second layer of water-insoluble polymers, about 80 to 10 percent by weight of the second layer of water-soluble osmopolymers, and about 5 to 50 percent by weight of the second layer of osmagents. The ratio of the first layer (drug-layer) to the second layer (push-layer) in the preparation should be about 1:0.5˜3, and preferably about 1:0.5˜1.5, more preferably 1:0.8˜1.2, and most preferably 1:1.

The controlled release composition contains polyvinylpyrrolidone homopolymers and/or copolymers as the major inert ingredient, and obviates the disadvantages of heat sensitivity and lengthy lag of drug release associated with the use of polyethyleneoxide (PEO) as the major inert ingredient. We found that using polyvinylpyrrolidone homopolymers and/or copolymers as the carriers in nifedipine controlled release tablets is surprisingly advantageous over PEO at least for several reasons. The controlled release tablets, using polyvinylpyrrolidone homopolymers and/or copolymers as a drug carrier, have a short lag time after administration to appearance of the desired physiological effect in the body. Furthermore, polyvinylpyrrolidone homopolymers and/or copolymers are more heat stable than PEO. For example, the glass transition temperature of Povidone (a PVP homopolymer) ranges from 130° C. to 176° C., depending on the particular molecular weight. The T_{g} of Povidone (Plasdone K-90) is 174° C., and the T_{g} of Copovidone (Plasdone S-630) (a PVP copolymer) is 105° C. So special conditions are not required in the preparation of the osmotic pump device and during storage. The drug using these carriers have better stability, and can exhibit better control of release of the nifedipine.

The present invention will be described in terms of a two-layer osmotic pump device, typically, in the form of a tablet. The term “layer” is used for ease of description, but it is understood that a layer may be a compartment containing the described materials. One of the layers is the drug containing layer or drug layer containing glipizide and the carrier as well as other ingredients as described herein. The other layer is the push layer which contains various kinds of osmopolymers, which are water-soluble hydrophilic polymers that, when dissolved, produce osmotic pressure in the drug layer. The push layer also contains water-insoluble polymers, which have high water absorption speed and high water absorption capacity so that they swell intensively when exposed to water. The swell of these water-insoluble polymers can have a mechanical push force on the drug release layer. Either or both of the drug layer and the push layer may contain an osmagent, also called an osmotic solute.

The glipizide controlled release tablets preferably have a membrane coating comprising semi-permeable material. It is permeable to water or other body fluid existing in the gastrointestinal tract, but it is impermeable to glipizide. The semi-permeable material includes, but is not limited to, cellulose polymers, such as, cellulose acetate, ethyl cellulose, cellulose diacetate, cellulose triacetate and the like.

The osmotic pump device preferably is a tablet form having at least one passageway communicating the drug-layer with the exterior for drug release. The passageway can be achieved by drilling, usually by laser, from the exterior through to the drug-layer. The size of the passageway is proportional to the drug release rate. A useful diameter of the passageway is about 0.2˜1.2 mm, preferably 0.4˜1.1 mm, more preferably 0.6˜1.0 mm.

The Push Layer

The push-layer comprises about 10 to 80 percent by weight of the push-layer of osmopolymers, about 10 to 80 percent by weight of the push-layer of water-insoluble polymers, and about 5-50% percent by weight of the push-layer of osmagents. The amount of water-insoluble polymers is preferably about 20˜60%, more preferably 20˜45%; the amount of the osmopolymers is preferably about 30˜70%, more preferably 40˜60%; and the amount of the osmagents is preferably about 8˜30%.

The insoluble polymers contained in the push layer are insoluble in water but have excellent rates of water-absorption and water-absorbing capacity. They swell rapidly and intensively after absorbing water to cause the pushing force. These polymers include sodium starch glycolate, low-substituted hydroxypropyl cellulose, crosslinked carboxylmethyl cellulose sodium and mixtures of two or more thereof. Experimental results of the cumulative release of drug (%) after 24 hours related to the weight of insoluble polymer in a tablet are shown in Table 1.

TABLE 1
Drug release

| | | Cumulative |
|---|---|---|
| | | drug release |
| | Amount | rate for |
| Water-insoluble polymers | (mg/tablet) | 16 hours (%) |
| sodium starch glycolate | 30 | 101.7 |
| low-substituted hydroxypropyl cellulose | 100 | 91.7 |
| crosslinked carboxylmethyl cellulose | 100 | 92.4 |
| sodium | | |
| sodium starch glycolate & low-substituted | 20 + 50 | 96.3 |
| hydroxypropyl cellulose | | |
| sodium starch glycolate & crosslinked | 20 + 50 | 97.9 |
| carboxylmethyl cellulose sodium | | |
| sodium starch glycolate & low-substituted | 25 + 20 + 20 | 99.1 |
| hydroxypropyl cellulose & crosslinked | | |
| carboxylmethyl cellulose sodium | | |

Based on these results, the optimal insoluble polymer is sodium starch glycolate.

The osmopolymers contained in the push layer are typically acrylic acid homopolymers and/or copolymers, hydroxypropylmethyl cellulose, polyvinypyrrolidone homopolymers, polyvinylpyrrolidone copolymers, or mixtures of two or more thereof. Useful acrylic homopolymers and/or copolymers are Carbomers. Commercially available Carbomers are typically homopolymers of acrylic acid, crosslinked with an allyl ether pentaerythritol, allyl ether of sucrose, or allyl ether of propylene.

The osmagents contained in the push layer are typically salts, organic acids and/or saccharides. Some useful salts are sodium chloride, potassium chloride, magnesium chloride, potassium sulphate, sodium sulphate and/or magnesium sulphate. Useful acids are ascorbic acid and/or tarraric acid. Typical saccharides are mannitol, sorbitol, xylitol, glucose and/or sucrose. Salts are the preferable osmagents and sodium chloride is most preferable.

The Drug Layer

The carriers in the drug layer carrying glipizide are polyvinylpyrrolidone homopolymers and/or copolymers. Usually they are about 40˜99% by weight of the drug-layer, and preferably 50˜90%, and more preferably 65˜85%.

A useful PVP polymer is Povidone, a synthetic homopolymer of linear 1-vinyl-2-pyrrolidone groups with a molecular weight in the range of about 1,000 to 3,000,000, typically about 1,300,000. A preferred polyvinylpyrrolidone copolymer is Copovidone, a copolymer of 1-vinyl-2-pyrrolidone and vinyl acetate in the mass proportion of from 1:10 to 10:1, such as the proportion of 7:3, 3:2, 5:5 or 3:7, wherein the proportion of 3:2 is preferable.

Both the drug layer and push layer may also contain a glidant such as silicon dioxide, a lubricant such as magnesium stearate and a colorant for distinguishing push layer and drug layer such as an inorganic stain. They may also contain other components such as hydrophilic materials for enhancing drug sustained release, diluters, adhesives and solvent. The drug-layer may also comprise one or more osmagents which are typically salts, such as NaCl, saccharides, such as, lactose, mannitol, glucose, sucrose or fructose, or acids, such as, ascorbic acid or tarraric acid.

Colorants may in the drug layer and/or the push layer and may comprise a mixture of two or more inorganic oxide colorants, such as red ferric oxide, yellow ferric oxide, purple ferric oxide, or black ferric oxide.

There may also other optional constituents in either or both layers, such as plasticizers, light blockers and pore formers. The plasticizers may be one or a mixture of two or more known plasticizers, such as diethyl phthalate, ethyl phthalate, triethyl citrate, or polyglycol. The light blockers may be one or a mixture of two or more, such as titanium dioxide, talc, and/or silicon dioxide. The pore formers may be one or a mixture of two or more, such as glycerin, propylene glycol, polyvinyl alcohol, or water-soluble inorganic salts.

Preparation

A method of preparing a glipizide controlled release composition is also provided.

Preparation of the drug-layer: Preferably working in dark conditions, first the nifedipine and 40˜99 percent of polyvinylpyrrolidone homopolymers and/or copolymers are sieved through a 60-mesh sieve, and then evenly mixed. By evenly mixed it is meant that portions are gradually added together to a mixture, with each portion consisting about half PVP and half glipizide. The mixture is added into a fluid-bed, and sprayed with alcohol water solution of a concentration of no less than about 40%. The wet mixture is granulated, dried and preferably magnesium stearic acid (and other optional ingredients) is added and evenly mixed.

Preparation of the push-layer: About 10 to 80 percent by weight of push-layer of osmopolymers, about 10 to 80 percent of water-insoluble polymers, and the osmagents are sieved through a 60-mesh sieve, then evenly mixed, preferably with adhesive, colorant and silicon dioxide acting as glidant (along with other optional ingredients). The mixture is added into a fluid-bed and alcohol solution with concentration of no less than about 40% is added. The mixture is evenly mixed.

The alcohol concentration in water solution utilized in the preparations above is typically about 40˜100%, preferably 60˜95%, and more preferably 75˜95%.

The inactive ingredients in the drug layer may also contain optional ingredients such as magnesium stearate, colorants, polyvinylpyrrolidone homopolymers and/or copolymers, osmagents and/or silicon dioxide. The inactive ingredients in the push layer may also contain adherents, colorants and/or glidants. These inactive ingredients can be sieved and mixed along with the other ingredients of the respective layers.

After the mixtures comprising the drug and push layers are formed, preferably working in the dark, the tablet is formed as follows. The mixture comprising one layer is pressed into the desired form, and then the mixture to form the other layer is applied to it and pressed together into a two-layer tablet core. Preferably the tablet core composition is coated with a semi-permeable membrane and dried, typically for about 3 hours at around 45° C. Then a passageway is drilled by machine or laser in the semi-permeable membrane adjacent to the drug-layer. A useful diameter of the passageway is about 0.9 mm. Typically an anti-damp film is coated over the semi-permeable membrane, and dried for about 3 hours at 45° C.

The solvent used for applying the coating of the semi-permeable membrane may typically be acetone, water, alcohol, dichloride, methanol, isopropylalcohol, or a mixture of two or more thereof. Acetone is preferred. For example, cellulose acetate (or other cellulose derivative) and/or diethyl phthalate (DEP) is dissolved in acetone to form a coating solution and it is used to coat the tablets on a conventional coating machine. An anti-damp film may be applied that not only protects the tablet from moisture, but can also provide a color mark for the tablet.

The technology used for passageway drilling and the membrane or film coating is well known in the pharmaceutical field.

The daily dosage of glipizide may be determined on a case-by-case basis keeping within an amount that is pharmaceutically effective for therapy or prevention of a particular disease or condition. Hence, tablets containing such amounts designed to deliver the payload of the desired daily dosage of the drug within 24 hours by sustained release would suffice. Typically, an osmotic pump controlled release tablet for once daily use will contain about 5 to 20 mg of dlipizide.

The following examples are illustrations of the present invention, but they should not be considered as limiting the scope of the invention in any way.

### EXAMPLE 1

Prescription:
(1) Drug layer (per tablet):

| | | |
|---|---|---|
| Glipizide | 5 | mg |
| Povidone (Plasdone K-90) | 20 | mg |
| Copovidone (Plasdone S630) | 69 | mg |
| Yellow ferric oxide | 0.05 | mg |
| Magnesium stearate | 0.75 | mg |
| Silicon dioxide | 0.5 | mg |

(2) Push layer (per tablet):

| | | |
|---|---|---|
| Sodium starch glycolate | 30 | mg |
| HPMC (K15M) | 14 | mg |
| Carbomer (971PNF) | 10 | mg |
| Sodium chloride | 30 | mg |
| Copovidone (Plasdone S630) | 15 | mg |
| Red ferric oxide | 0.95 | mg |
| Magnesium stearate | 0.48 | mg |
| Silicon dioxide | 0.5 | mg |

(3) Semi permeable membrane coating solution (amount used per 1000
tablets)

| | | |
|---|---|---|
| Cellulose acetate | 45 | g |
| Diethyl phthalate | 2.5 | g |
| Acetone water solution | 2000 | ml |

(4) Film coating solution:

| | |
|---|---|
| OPADRY II White | sufficient amount |
| (85G68918) | |

The Povidone (Plasdone K-90) can be substituted with other amounts of Plasdone K-90D, Plasdone K-15, Plasdone K-30, Plasdone K-60, Plasdone K-120, or a mixture of two or more of these. The HPMC (K 15M) can be substituted with other amounts of HPMC K4M, HPMC K100 M, HPMC K100LV or a mixture of two or more of these.

Preparation Method:

1. Preparation of the drug-layer granules:

Povidone (Plasdone K-90) and Copovidone (Plasdone S630) and silicon dioxide are sieved through a 60-mesh screener and homogeneously mixed with Glipizide and yellow ferric oxide. Then the solid mixture is added into a fluid bed granulator, and an alcohol water solution of about 75% is sprayed in to granulate (the temperature of the inlet is 30˜70° C.). Water content, drug content, content uniformity and related compounds are determined after drying. Then magnesium stearate is added and mixed evenly to obtain the drug-layer granules.

2. Preparation of the push-layer granules:

First, all the ingredients are sieved through a screener of 60 mesh separately, then sodium starch glycolate, HPMC, Carbomer, NaCl, Copovidone (Plasdone S630), red ferric oxide are mixed together with silicon dioxide. The mixture is added into a fluid-bed granulator, then an alcohol water solution of about 75% is sprayed in to granulate followed by drying, then water percent is determined. Then magnesium stearate is added and mixed evenly to obtain the push-layer granules.

3. Tablet pressing:

The two-layer tablet cores are formed by pressing together the two of granule formulations. The tablet diameter is 8 mm. Drug hardness and content and content uniformity of the two-layer tablets are determined.

4. Tablet coating with semi-permeable membrane:

The semi-permeable membrane is coated over the tablet core followed by drying at 45° C. for about 3 hours. The acetone residue amount is determined.

5. An orifice with diameter of 0.9 mm is drilled in the wall adjacent to the drug-layer by machine or laser. The drug release is assayed.

6. Tablet coating with anti-damp film:

The tablet after drilling is coated with the anti-damp film followed by drying at 45° C. for about 3 hours. Then the tablets are packed for storage or shipping.

### EXAMPLE 2

Prescription:
(1) Drug layer (per tablet):

| | | |
|---|---|---|
| Glipizide | 5 | mg |
| Povidone (Plasdone K-90) | 40 | mg |
| Copovidone (Plasdone S630) | 83 | mg |
| Yellow ferric oxide | 0.05 | mg |
| Magnesium stearate | 0.75 | mg |
| Silicon dioxide | 0.5 | mg |

(2) Push layer (per tablet):

| | | |
|---|---|---|
| Sodium starch glycolate | 70 | mg |
| HPMC (K15M) | 5 | mg |
| Carbomer (971PNF) | 15 | mg |
| Sodium chloride | 10 | mg |
| Copovidone (Plasdone S630) | 15 | mg |
| Red ferric oxide | 0.95 | mg |
| Magnesium stearate | 0.48 | mg |
| Silicon dioxide | 0.5 | mg |

The process and manufacturing technology of semi permeable membrane and anti-damp film coating are the same as that of Example 1.

### EXAMPLE 3

Prescription:
(1) Drug layer (per tablet):

| | | |
|---|---|---|
| Glipizide | 5 | mg |
| Povidone (Plasdone K-90) | 55 | mg |
| HPMC (K15M) | 45 | mg |
| Yellow ferric oxide | 0.05 | mg |
| Magnesium stearate | 0.75 | mg |
| Silicon dioxide | 0.5 | mg |

(2) Push layer (per tablet):

| | | |
|---|---|---|
| Sodium starch glycolate | 70 | mg |
| HPMC (K15M) | 5 | mg |
| Carbomer (971PNF) | 15 | mg |
| Sodium chloride | 10 | mg |
| Copovidone (Plasdone S630) | 15 | mg |
| Red ferric oxide | 0.95 | mg |
| Magnesium stearate | 0.48 | mg |
| Silicon dioxide | 0.5 | mg |

The process and manufacturing technology of semi permeable membrane and anti-damp film coating are the same as that of Example 1.

### EXAMPLE 4

Prescription:
(1) Drug layer (per tablet):

| | | |
|---|---|---|
| Glipizide | 5 | mg |
| Povidone (Plasdone K-90) | 85 | mg |
| Copovidone (Plasdone S630) | 155 | mg |
| Yellow ferric oxide | 0.05 | mg |
| Magnesium stearate | 0.75 | mg |
| Silicon dioxide | 0.5 | mg |

(2) Push layer (per tablet):

| | | |
|---|---|---|
| Crosslinked carboxylmethyl cellulose sodium | 150 | mg |
| HPMC (K15M) | 35 | mg |
| Carbomer (971PNF) | 25 | mg |
| Sodium chloride | 45 | mg |
| Copovidone (Plasdone S630) | 35 | mg |
| Red ferric oxide | 0.95 | mg |
| Magnesium stearate | 0.48 | mg |
| Silicon dioxide | 0.5 | mg |

The process and manufacturing technology of semi permeable membrane and anti-damp film coating are the same as that of Example 1.

### EXAMPLE 5

Prescription:
(1) Drug layer (per tablet):

| | | |
|---|---|---|
| Glipizide | 5 | mg |
| Povidone (Plasdone K-90) | 30 | mg |
| Copovidone (Plasdone S630) | 15 | mg |
| Carbomer (971PNF) | 30 | mg |
| Yellow ferric oxide | 0.05 | mg |
| Magnesium stearate | 0.75 | mg |
| Silicon dioxide | 0.5 | mg |

(2) Push layer (per tablet):

| | | |
|---|---|---|
| Sodium starch glycolate | 25 | mg |
| HPMC (K15M) | 25 | mg |
| Sodium chloride | 39 | mg |
| Copovidone (Plasdone S630) | 35 | mg |
| Red ferric oxide | 0.95 | mg |
| Magnesium stearate | 0.48 | mg |
| Silicon dioxide | 0.5 | mg |

The process and manufacturing technology of semi permeable membrane and anti-damp film coating are the same as that of Example 1.

### EXAMPLE 6

Prescription:
(1) Drug layer (per tablet):

| | | |
|---|---|---|
| Glipizide | 5 | mg |
| Povidone (Plasdone K-90) | 32 | mg |
| Copovidone (Plasdone S630) | 20 | mg |
| Yellow ferric oxide | 0.05 | mg |
| Magnesium stearate | 0.75 | mg |
| Silicon dioxide | 0.5 | mg |

(2) Push layer (per tablet):

| | | |
|---|---|---|
| Sodium starch glycolate | 55 | mg |
| Carbomer (971PNF) | 20 | mg |
| Sodium chloride | 11 | mg |
| Copovidone (Plasdone S630) | 23 | mg |
| Red ferric oxide | 0.95 | mg |
| Magnesium stearate | 0.48 | mg |
| Silicon dioxide | 0.5 | mg |

The process and manufacturing technology of semi permeable membrane and anti-damp film coating are the same as that of Example 1.

### EXAMPLE 7

Prescription:
(1) Drug layer (per tablet):

| | | |
|---|---|---|
| Glipizide | 5 | mg |
| Povidone (Plasdone K-90) | 63 | mg |
| Copovidone (Plasdone S630) | 34 | mg |
| Yellow ferric oxide | 0.05 | mg |
| Magnesium stearate | 0.75 | mg |
| Silicon dioxide | 0.5 | mg |

(2) Push layer (per tablet):

| | | |
|---|---|---|
| Sodium starch glycolate | 21 | mg |
| HPMC (K15M) | 30 | mg |
| Carbomer (971PNF) | 10 | mg |
| Sodium chloride | 35 | mg |
| Copovidone (Plasdone S630) | 35 | mg |
| Red ferric oxide | 0.95 | mg |
| Magnesium stearate | 0.48 | mg |
| Silicon dioxide | 0.5 | mg |

The process and manufacturing technology of semi permeable membrane and anti-damp film coating are the same as that of Example 1.

### EXAMPLE 8

Prescription:
(1) Drug layer (per tablet):

| | |
|---|---|
| Glipizide | 5 mg |
| Povidone (Plasdone K-90D) | 20 mg |
| Copovidone (Plasdone S630) | 61 mg |

(2) Push layer (per tablet):

| | |
|---|---|
| Sodium starch glycolate | 35 mg |
| HPMC (K15M) | 30 mg |
| Carbomer (971PNF) | 10 mg |
| Sodium chloride | 22 mg |
| Copovidone (Plasdone S630) | 15 mg |
| Red ferric oxide | 1.1 mg |

The process and manufacturing technology of semi permeable membrane and anti-damp film coating are the same as that of Example 1.

Preparation Method:

1. Preparation of the drug-layer granules:

All ingredients are sieved through a 60-mesh screener. Then Povidone (Plasdone K-90D), Copovidone (Plasdone S630) and glipizide are homogeneously mixed. The solid mixture is added into a fluid bed granulator, and an alcohol water solution of about 75% is sprayed in to granulate. Water content, drug content, content uniformity are determined after drying.

2. Preparation of the push-layer granules:

All the ingredients are sieved through a screener of 60 mesh separately, and then are homogeneously mixed. The mixture is added into a fluid-bed granulator, then an alcohol water solution of about 75% is sprayed in to granulate followed by drying, then water percent is determined.

3. Tablet pressing: refer to Example 1.

4. Tablet coating with semi-permeable membrane: refer to Example 1.

5. An orifice with diameter of 0.9 mm is drilled into the wall adjacent to the drug-layer by machine or laser. The drug release is assayed.

### EXAMPLE 9

The drug release of tablets made according to the present invention compared to a commercial product (Glucotrol® XL) is determined in different media with different pH values.

(1) sodium dodecylsulfete chloride solution of 0.5% (pH=1.2)

(2) buffer solution of sodium dodecylsulfete of 0.5% dissolved in acetic acid-sodium acetate solution (pH=4.5);

(3) simulated gastric fluid without pancreatin (pH6.8).

Assay method: Drug Release Test 1 (Appendix X D in Part 2 of China Pharmacopeia 2005th)

Apparatus: paddle of Apparatus 2 (Appendix X C in Part 2 of China Pharmacopeia 2005th): 50 rotations per minute.

Medium: 900 ml as described above

Times: 2, 4, 6, 8, 10, 12 and 16 hours

At the end of each specified test interval, 8 ml is sampled, and filtered by 0.45 μm micropore filter membrane. Then 8 ml of fresh medium is immediately supplemented. The UV absorbance is determined at the wavelength of 276 nm. About 50 mg of glipizide reference is transferred and accurately weighed into a 100-ml volumetric flask. Then 20 ml of methanol are added and the mixture is sonicated to dissolve the glipizide. The mixture is diluted with methanol and mixed to obtain a stock solution. Quantitative dilution of the stock solution with different media provides reference solutions having suitable known concentrations of glipizide, as follows

| No. of | | Concentration |
|---|---|---|
| reference | | of Glipizide |
| solution | Diluting method | (μg/ml) |
| 1# | dilute 1 ml of stock solution to 200 ml with | 2.5 |
| | medium solution | |
| 2# | dilute 1 ml of stock solution to 100 ml with | 5.0 |
| | medium solution | |
| 3# | dilute 3 ml of stock solution to 100 ml with | 15.0 |
| | medium solution | |
| 4# | dilute 25 ml of 1# reference solution to | 1.25 |
| | with medium solution | |
| 5# | dilute 25 ml of 3# reference solution to | 7.5 |
| | 50 ml with medium solution | |
| 6# | dilute 25 ml of 4# reference solution to | 0.625 |
| | 50 ml with medium solution | |

UV absorbance of reference solution 6#, 4#, 1#, 2#, and 5# are determined at 276 nm as directed above. A working curve is formed, and the drug amount released at each specified test interval is calculated according to the working curve. The results are shown in Table. 2

TABLE 2
Drug release data of Glipizide CR tablets (n = 6)

| | Drug release (%) | | | | | | | |
|---|---|---|---|---|---|---|---|---|
| Media | 2 h | 4 h | 6 h | 8 h | 10 h | 12 h | 16 h | |
| Test | pH 1.2 | 3.99 | 22.14 | 40.49 | 59.45 | 76.89 | 93.21 | 100.85 |
| (Ex. 9) | pH 4.5 | 6.43 | 24.02 | 43.56 | 62.78 | 79.76 | 95.04 | 103.56 |
| | pH 6.8 | 7.59 | 23.61 | 41.39 | 60.17 | 78.63 | 93.04 | 103.23 |
| Reference | pH 1.2 | 0.56 | 17.48 | 35.25 | 53.22 | 71.52 | 87.51 | 98.26 |
| product | pH 4.5 | 1.55 | 19.21 | 37.34 | 56.63 | 73.95 | 92.02 | 103.49 |
| | pH 6.8 | 0.56 | 19.55 | 37.95 | 57.09 | 75.51 | 92.68 | 102.86 |

The results show that the drug release data of tablets made according to the present invention and the commercial product both meet the standard requirements. However, compared to the commercial product, the tablets made according to the present invention take effect in a shorter time and exhibit better drug release at the final stage (16 hours).

### EXAMPLE 10

Studies were conducted on healthy volunteers of pharmacokinetics and bioavailability of formulations prepared according to the present invention compared to commercial formulations.

Method: A randomized crossover study according to body weight index, 2-period, under fasted state. 24 healthy male volunteers were crossed and randomized to administer a single-dose of 5 mg of glipizide controlled-release tablet (test drug, prepared according to the present invention) and Glucotrol® XL (reference drug), respectively. The blood concentrations were determined by LC-MS/MS, and pharmacokinetic parameters of two formulations and relative bioavailability of test drug were calculated with the software 3P97, emphasizing on the difference of blood concentrations of the respective drugs during the initial stage after administration to indicate the difference of time lag of drug release in vivo.

The 24 healthy male volunteers randomized to 2 groups, 12 volunteers per group, who were fasting after supper of the day before study and were allowed to take food 2 hours later after administration. They were crossed to orally administer 5 mg of test drug and reference drug, respectively. They took standard food for lunch and were allowed to take some water during the study. Then 4 ml of blood sample was taken from each volunteer pre-dose and at 0.5, 1.0, 2.0, 3.0, 4.0, 6.0, 8.0, 10.0, 12.0, 16.0, 24, 36, and 48 hours after dosing. The samples were centrifuged and placed under −60° C. condition for determination.

After checking data, blood concentrations of each time point were calculated with the weighted regression method and then data were collected with EXCEL software. Software of 3P97 was used to calculate the pharmacokinetic parameters.

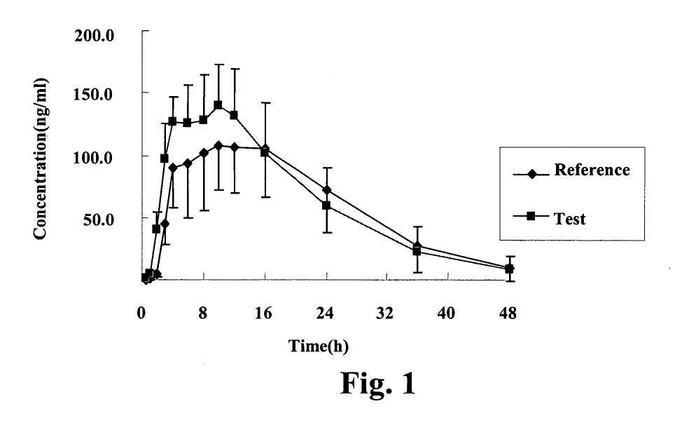

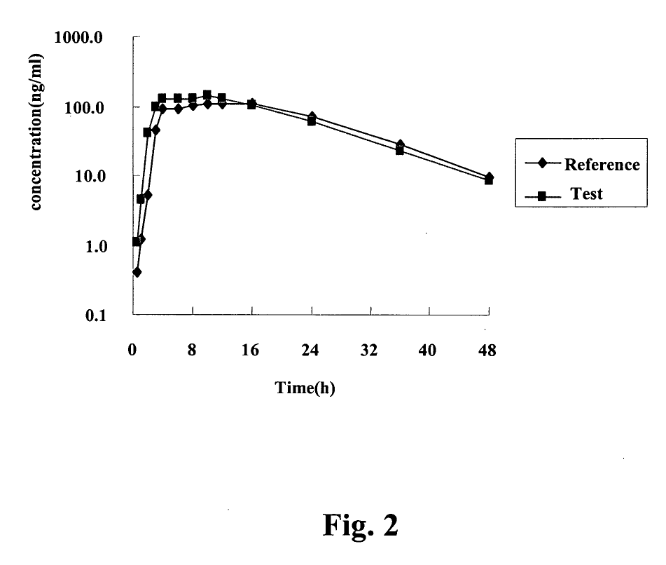

The curve of mean blood concentration to time is shown in FIG. 1. The log of the mean blood concentration vs. time is shown in FIG. 2. The changing trends of two groups of curves (test and reference) are seen to be basically consistent. The test formulation and reference formulation both show the properties of controlled release. However, the initial blood concentration after administration of test formulation is obviously higher than that of the reference formulation, and the peak time of test formulation is shorter than that of reference formulation. The Tₘₐₓ of the reference formulation is 10.67±5.13 hours, while that of test formulation is 9.08±2.76 hours, which shows that time lag of drug release of the test formulation is obviously shorter than that of the reference formulation.

What is claimed is:

1. An osmotic pump device for administration of glipizide at a controlled rate into a biological environment comprising:
a core comprising a first layer containing a pharmaceutically effective amount of glipizide and about 40 to 99 percent by weight of said first layer of carrier polymers comprising hydrophilic homopolymers and/or copolymers of polyvinylpyrrolidone, and a second layer comprising about 10 to 80 percent by weight of said second layer of water-insoluble polymers, about 80 to 10 percent by weight of said second layer of water-soluble osmopolymers, and about 5 to 50 percent by weight of said second layer of osmagents.

2. A device according to claim 1 wherein the ratio of weight of said first layer to said second layer is in the range of about 1:0.5 to 1:3.

3. A device according to claim 1 wherein the said polyvinylpyrrolidone homopolymers comprise a homopolymer of linear 1-vinyl-2-pyrrolidone groups.

4. A device according to claim 1 wherein said polyvinylpyrrolidone copolymers comprise a copolymer of 1-vinyl-2-pyrrolidone and vinyl acetate in the mass proportion of about 1:10 to 10:1.

5. A device according to claim 1 wherein said core further comprises an adhesive, lubricant, a glidant, and/or a colorant.

6. A device according to claim 1 wherein said water-insoluble polymers in said second layer are selected from the group consisting of sodium starch glycolate, low-substituted hydroxypropyl cellulose, crosslinked carboxylmethyl cellulose sodium and mixtures of two or more thereof.

7. A device according to claim 1 wherein said osmopolymers in said second layer are selected from the group consisting of acrylic acid polymers, acrylic acid copolymers, hydroxypropylmethyl cellulose, polyvinypyrrolidone homopolymers, polyvinylpyrrolidone copolymers and mixtures of two or more thereof.

8. A device according to claim 7 wherein the said acrylic acid polymers and/or copolymers comprise homopolymers of acrylic acid, crosslinked with an allyl ether pentaerythritol, allyl ether of sucrose, or allyl ether of propylene.

9. A device according to claim 1 wherein said osmagents in said second layer are selected from the group consisting of water soluble inorganic salts, organic acids, saccharides and mixtures of two or more thereof.

10. A device according to claim 9 wherein the said osmagents comprise salts.

11. A device according to claim 10 wherein said salts are selected from the group consisting of sodium chloride, potassium chloride, magnesium chloride, potassium sulphate, sodium sulphate and magnesium sulphate.

12. A device according to claim 9 wherein said saccharides are selected from the group consisting of mannitol, sorbitol, xylitol, glucose and sucrose.

13. A device according to claim 9 wherein said acids are selected from the group consisting of ascorbic acid and tarraric acid.

14. A device according to claim 1 further comprising a wall surrounding said core comprising a semi-permeable material permeable to the passage of an exterior fluid and substantially impermeable to the passing of glipizide

15. A device according to claim 14 wherein said semi-permeable material comprises cellulose polymers.

16. A device according to claim 14 further comprising a passageway in said wall communicating with said first layer and the exterior of said device for delivery of said glipizide from said device.

17. A device according to claim 16 wherein said passageway is of the diameter of about 0.2 to 1.2 mm.

18. A method of preparation of said core of a device according to claim 1 comprising:
preparing the composition for said first layer by
a) separately passing glipizide, said carrier polymers and optional ingredients desired for said first layer through a 60-mesh sieve;
b) mixing said glipizide with said polymer carriers and said optional ingredients for said first layer to form a first ingredient mixture;
c) spraying said first ingredient mixture with an alcohol solution to form a first wet mixture;
d) granulating said first wet mixture;
and preparing the composition for said second layer by
i) separately passing said water-insoluble polymers, said water-soluble osmopolymers, said osmagents and optional ingredients for said second layer through a 60-mesh sieve;
ii) mixing said osmopolymers, said water-insoluble polymers, said osmagents and said optional ingredients for said second layer to form a second ingredient mixture;
iii) spraying said second ingredient mixture with alcohol solution to form a second wet mixture;
iv) granulating said second wet mixture;
1) pressing at least a portion of said granulated first wet mixture from step (d) to form said first layer;
2) applying at least a portion of said granulated second wet mixture from step (iv) onto said first layer from step (1) to form a core precursor;
3) pressing said core precursor to form said core comprising said first and second layers.

19. A method according to claim 18 further comprising the step 4) of coating said core with a membrane of semi-permeable polymers.

20. A method according to claim 19 wherein said semi-permeable polymers comprise cellulose polymers.

21. A method according to claim 18 wherein said optional ingredients of said second layer comprise an adhesive, lubricant, glidant, and/or colorant.

22. A method according to claim 19 or 20 further comprising the step of applying an anti-damp film over said membrane.
